(19) United States
(12) Patent Application Publication
(10) Pub. No.: US 2002/0068918 A1
(43) Pub. Date: Jun. 6, 2002

(54) Tampon string accessory apparatus

(76) Inventor: Maxie A. Durel-Crain, Lafayette, LA (US)

Correspondence Address: GARVEY SMITH NEHRBASS & DOODY, LLC, THREE LAKEWAY CENTER, 3838 NORTH CAUSEWAY BLVD., SUITE 3290, METAIRIE, LA, 70002

(21) Appl. No.: 09/975,747

(22) Filed: Oct. 11, 2001

Related U.S. Application Data
(63) Continuation-in-part of application No. 09/285,616, filed on Apr. 3, 1999.

(51) Int. Cl. A61F 13/15; A61F 13/20

(52) U.S. Cl. 604/385.18

(57) ABSTRACT

An accessory in the form of a tab (24), ring (25), tassel (26) or puff ball (28) is attached to a tampon string (16) to facilitate the tactile location and manipulation of the tampon string (16) during urination. The accessory may also help prevent the tampon string (16) from being retracted into the vagina. Additional embodiments are illustrated along with a method for extending the tampon string with the accessory attachment having the capability of being adhered to the user's body or undergarment.

### CROSS REFERENCE TO RELATED APPLICATIONS

Co-pending U.S. patent application Ser. No. 09/285,616, filed Apr.3 1999, is incorporated herein by reference. Priority of that application is hereby claimed

### STATEMENT REGARDING FEDERALLY SPONSORED RESEARCH OR DEVELOPMENT

Not applicable

### REFERENCE TO A “MICROFICHE APPENDIX”

Not applicable

### BACKGROUND OF THE INVENTION

1. Field of the Invention

This invention relates generally to feminine hygienic tampons having strings attached there to for withdrawal. More particularly, the invention relates to a method for attaching an accessory or tab to the string to facilitate locating and preventing dislocation of the tampon string.

2. General Background of the Invention

The tampon industry estimates that on average a women will use 11,400 tampons in her lifetime. Many of these tampon users are young, highly active women who become so accustomed to the presence of the tampon that they literally forget it is in place. Tampons are generally provided with a string which extends outside the vagina to assist in removal of the tampon from the body cavity. This string generally comprises a string looped around a portion of the tampon in a manner which produces a pair of string ends knotted together at their free end. Therefore, due to frictional migration and the expansion and contraction of the vagina, especially during physical activity, the tampon can get lodged near the back of the vagina or rotated in such a manner that the string is withdrawn into the vagina cavity. Since the string is not easily visible and not capable of being felt, women Odin forget to change the tampon every 4-6 hours as recommended by the manufacturer. On occasion some women forget and accidentally insert a new tampon without removing the previous one. Such action leads to serious complication. Some women who are not familiar with their bodies sometimes panic, fearing that the tampon is lost and that perhaps surgery will be required to retrieve the tampon. It is estimated that each gynecologist experiences the need to retrieve lost tampons an average of twice per month. Therefore, this is serious problem affecting thousands of women nationally every year. Tampons which are not removed regularly provide a breeding ground for staphylococcus Aureus, a bacterium which leads to a number of serious medical problems, such as toxic shock syndrome.

The tampon string is also a problem during urination. Most women, assuming they remember the tampon string at all, have a preference as to the question of whether to try and locate the short string or simply urinate on it thus leaving the string wet and subject to bacterial infection. Most tampon manufacturers provide a relatively short, string usually less than three inches (7.6 cm), making the movement of the string to a position out of the way during urination an awkward maneuver at best, thus it is simply easier to wet the string. Since most tampon strings are made of cotton, they absorb moisture readily and thus further provide a breeding ground for infectious bacteria. To make matters worse, women seldom wash their hands in public rest rooms. This phobia makes them reluctant to search for and handle the short, often moist, tampon string. It is therefore evident that the above problems need to be addressed in a manner which will allow women to retrieve a tampon more readily and become more aware of its presence.

The following U.S. Patents are incorporated herein by reference: 1,555,708; 3,037,506; 3,625,787; 3,749,094; 3,948,257; 4,332,251; 4,341,214; 4,490,894; 5,370,633; 5,458,589; 5,533,990; 5,566,435; 5,659,934; 5,674,239; 5,785,053; 5,800,338; and 5,807,372.

### BRIEF SUMMARY OF THE INVENTION

The apparatus of the present invention is a tampon with a means for preventing the tampon string from completely retreating into a user's vagina. Also provided is a means for preventing a tampon string from completely retreating into a user's vagina.

The present invention provides an accessory in the form of, for example, a tab, tassel or puff ball attached to a tampon string. Most preferably, the accessory is a tab comprising a piece of tape including a peel-off backing which, when removed, exposes a sticky surface. The accessory is intended to help the tampon user more easily locate and manipulate the tampon string during urination and may be more readily discerned if the string is being retracted into the vagina. Subsequent illustrations provide a method for extending the length of the tampon string with the accessory attachment and further having the optional capability of being adhered to the users body or undergarment. The intended purpose of the accessory is to help draw attention to the tampon and facilitate the location and manipulation of the tampon string during urination. The accessory further draws the user's attention to the presence of the tampon and, therefore, serves as a reminder to change the tampon frequently. The accessory also helps to prevent migration of the tampon string into the vagina.

The present invention also comprises a tampon with a longer than customary string. This longer string can be provided when the tampon is manufactured or it can be made by attaching an additional length of string to a tampon string of an already manufactured tampon. In either event, the longer string makes it easier for a woman to locate the free end of the tampon string.

### BRIEF DESCRIPTION OF THE SEVERAL VIEWS OF THE DRAWINGS

For a further understanding of the nature, objects, and advantages of the present invention, reference should be had to the following detailed description, read in conjunction with the following drawings, wherein like reference numerals denote like elements and wherein:

FIG. 1 is an isometric view of a tampon and insertion tube and a first embodiment of the present invention;

FIG. 2 is an isometric view of a tampon shown in phantom connected to a second embodiment of the present invention;

FIG. 3 is an isometric view of a tampon shown in phantom connected to another embodiment of the present invention;

FIG. 4 is an isometric view of a tampon and insertion tube and yet another embodiment of the present invention;

FIG. 5 is an isometric view of a tampon with an accessory of the present invention attached;

FIG. 6 is a partial isometric view of a woman, illustrating the present invention in use;

FIG. 7 shows the tampon with the tassel attached directly to the tampon string with no extension;

FIG. 8 shows the tampon with the pom-pom attached directly to the tampon string with no extension;

FIG. 9 shows the tampon with the tape attached directly to the tampon string with no extension;

FIG. 10 shows the tampon with a ring attached to an extension;

FIG. 11 shows the tampon with a ring attached directly to the tampon string with no extension;

FIG. 12 shows the tampon with a capsule attached to an extension;

FIG. 13 shows the tampon with a capsule attached directly to the tampon string with no extension;

FIG. 14 shows the tampon with an enlarged knot;

FIG. 15 shows the tampon with an increased width of the tampon string;

FIG. 16 shows a waist band to which the tampon string can attach;

FIG. 17 shows the tampon with an extra long string; and

FIG. 18 shows the tampon with the extra long string attached to a waist band around a woman's waist.

### DETAILED DESCRIPTION OF THE INVENTION

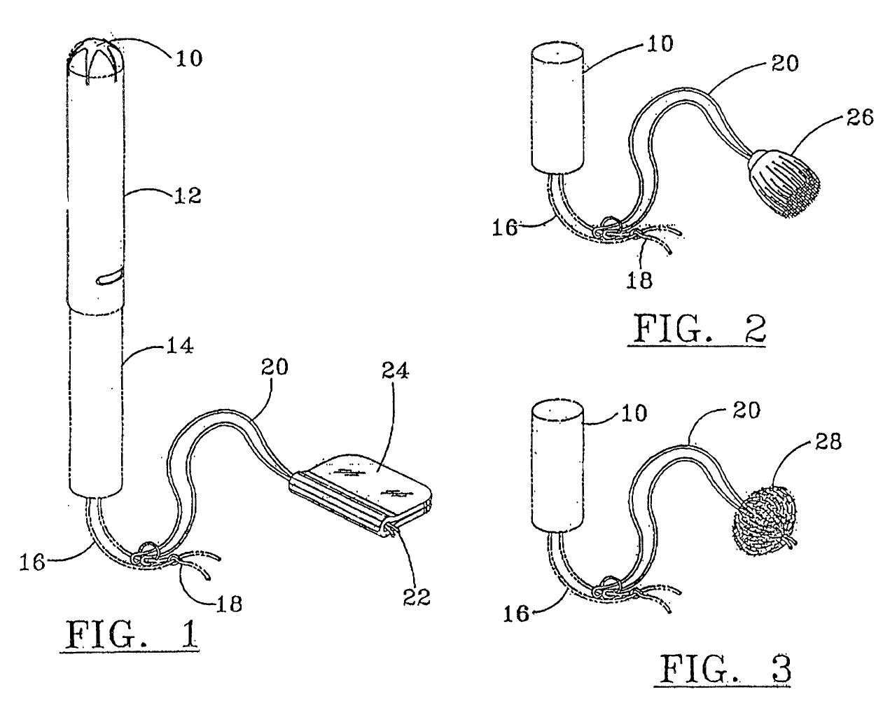

As illustrated in FIG. 1, tampons 10 are often supplied installed within an insertion tube 12 having a plunger portion 14. The tampon string attached to the tampon 10 extends longitudinally through the hollow tubular plunger 14 the string 16 generally comprising the two ends of the same string knotted at their free end 18. The string 16 usually is not more than four and one half inches to five inches (11.4-12.7 cm) in length. It is anticipated that since vast numbers of these tampons are already available, an attachment can advantageously be provided in the form of an extension string 20 which can be threadably looped through the existing string 16 behind the knot at the free end 18 in a manner whereby the two strings are connected, thus increasing the string length by, for example, between 3 and 6 inches (7.6-15.2 cm). The extension string 20 (which can include a loop as shown) is connected at its free end 22 to a tab 24 which is adhered or otherwise secured around a portion of the extension string 20. The tab 24 can be of any configuration, but preferably one which is not excessively bulky or irritating such as a length of cloth-backed tape folded in half or otherwise secured around the string. However, the tab is preferably sufficiently large to prevent retraction into the vagina as a result of frictional migration or other exertion. The extension string 20 extends the length of the string 16, usually adding about 6 to 7 inches (15.2-17.8 cm) in the overall length for an overall length of about 11 inches (27.9 cm), and results in an extended string 20 and a tab 24 which allows the user to more easily locate and position the string during urination.

As seen in FIG. 2, the string accessory maybe a tassel 26 or the like having several strands of thread which could be used at the end of the extension string 20. This embodiment provides a tactilely noticable body at the end of the string 20, yet the accessory will lie virtually flat and invisible under the user's garments.

The accessory seen in FIG. 3 may also be a non-absorbent puffball, such as a knit globe or spherical body 28 having a hollow core which folds along its equator, or other such soft, non-absorbent body which will tend to expand and collapse diametrically upon axial compression along its polar axis.

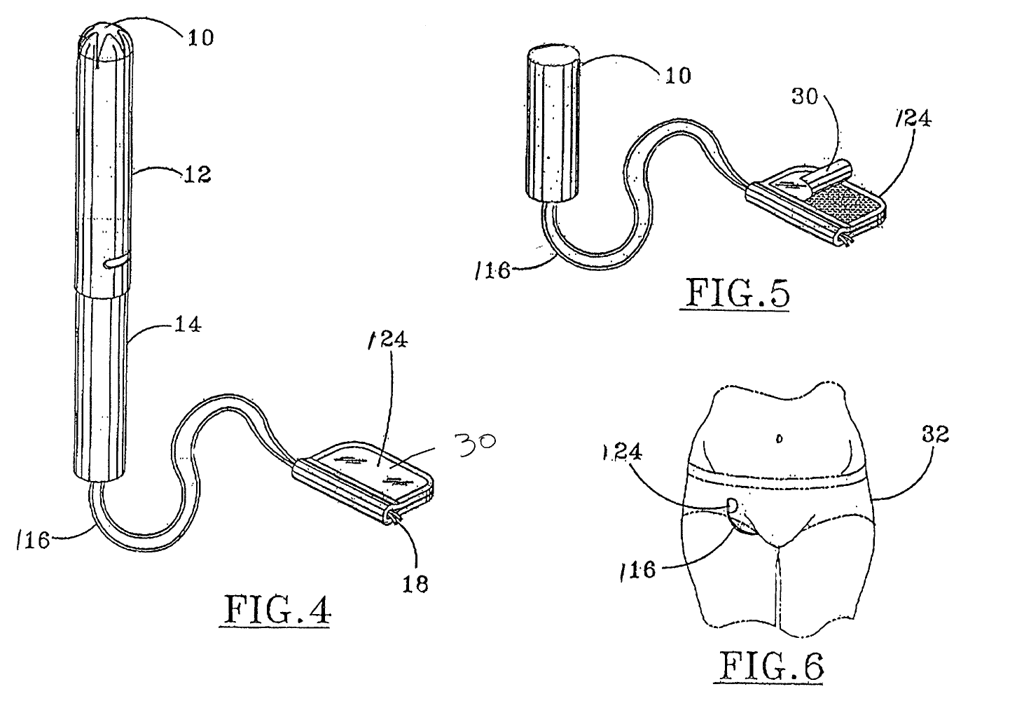

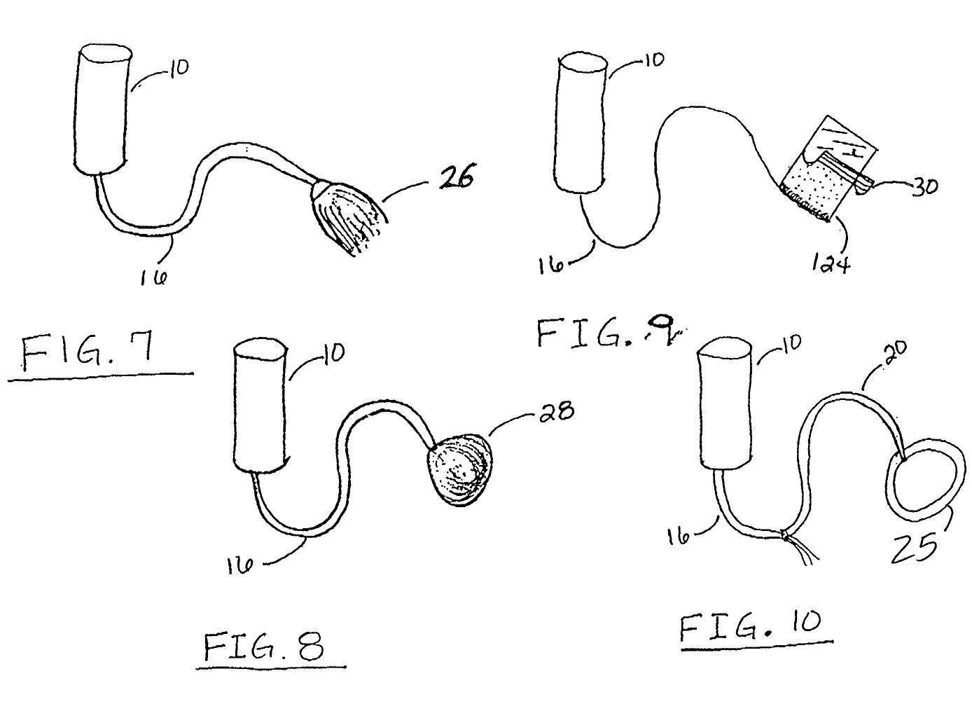

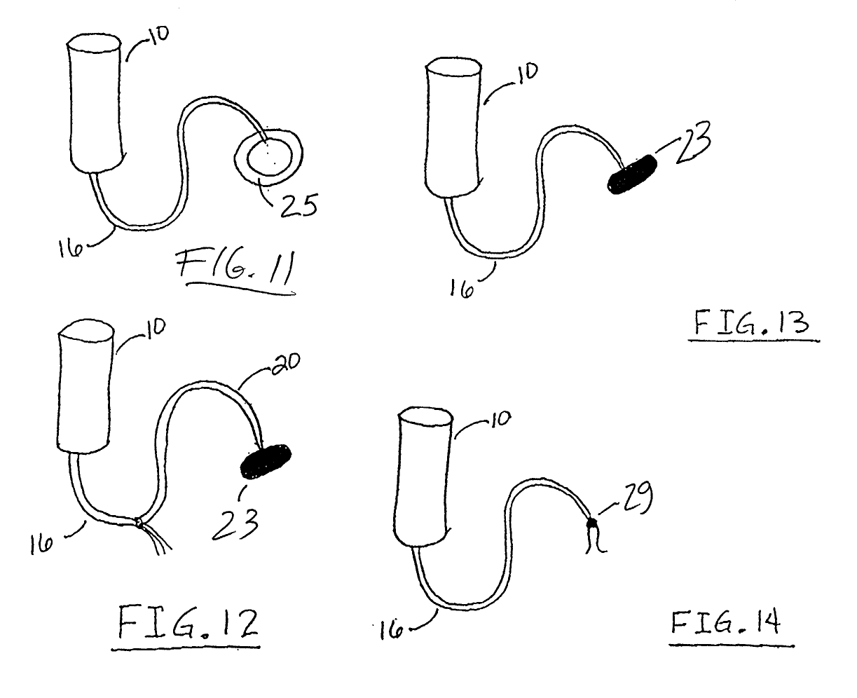

The tampon string 116 as illustrated in FIG. 4 can certainly be extended during manufacture, thus avoiding the need to provide an extension string 20. The tab 124 may also be attached during manufacture or provided as an optional accessory item in the form of a peel off tab which maybe folded in half around the knotted end 18 of the string 116 as shown, the sticky, peel-off sides folded to face each other and being adhered to the string 116.

It is also anticipated that the tab 124 maybe a double sided, soft cloth tape having a peel-off backing 30 as seen in FIGS. 4 and 5 which, when removed, provides a sticky surface which allows the tab 124 to be temporarily and removably adhered to a portion of the user's body or undergarment 32. Preferably the tab 124 would be adhered to the user's undergarment 32 as seen in FIG. 6 in the vicinity of the upper thigh normally covered by the user's undergarment 32.

In use, the accessories 24, 25, 26, 28, 124 are preferably sufficiently large to attract the user's attention any time the lower undergarment 32 is removed, thus reminding the user to change the tampon frequently. The string is preferably sufficiently long to allow the tampon user to readily locate the string 16, 20, 116 without searching and hold or position it to prevent saturation during urination.

The accessories 24, 25, 26, 28, 124 are preferably sufficiently large to prevent migration into the vagina, yet light and resilient enough to be virtually invisible when under the user's clothing.

When tab 24 is made of tape, it is preferably made of a hypo-allergenic tape, more preferably 3M's Microfoam tape, and most preferably Smith and Nephew's Hypafix Dressing Retention Sheet. The tape can be any reasonable thickness, preferably tape of no more than 1/32″ thickness (0.8 mm), and more preferably tape of no more than 1/128″ (0.2 mm) thickness. The length of the tape is preferably ¼″ to 3″ (0.64-7.6 cm), more preferably ½″ to 2″ (1.3-5.1 cm), and most preferably ¾″ to 1½″ (1.9-3.8 cm). The width of the tape is preferably ¼″ to 3″ (0.64-7.6 cm) to 2″ (0.85-5.1 cm), and most preferably ½″ to 1″ (1.3-2.5 cm)

The diameter of tassel 26 is dependent on the string diameter and the number of strings. Tassel 26 is preferably ½″ to 2″ (1.3-5.1 cm) long with approximately 1 to 34 strings which when doubled make 2 to 68 strings. Tassel 26 is more preferably ¾″ to 1½″ (1.9-3.8 cm) long wit approximately 10 to 40 strings which when doubled make 20 to 80 strings. Tassel 26 is most preferably 1″ to 1¼″ (2.5-3.2 cm) long with approximately 20 to 30 strings which when doubled make 40 to 60 strings.

The diameter of globe (pom-pom) 28 is preferably 10-20 mm, more preferably 10-16 mm, and most preferably 10-13 mm.

Tassel 26 or globe (pom-pom) 28 can be made of, for example, nylon, acrylic, or cotton, preferably acrylic or cotton, and most preferably cotton.

Ring 25 is preferably ½″ to 1½″ (1.3-3.8 cm) in diameter, more preferably ½″ to 1¼″ (1.33-03.2 cm) in diameter, and most preferably ¾″ to 1″ (1.9-2.5 cm) in diameter.

Ring 25 is preferably made out of any soft substance, more preferably made out of 3 M's Microfoam tape folded onto itself and cut into a ring, or made out of a satin cord, and most preferably is made out of a satin cord.

The length of string 20 is preferably ½″ to 8″ (1.3-20.3 cm), more preferably ¾″ to 5″ (1.91-12.7 cm), and most preferably 1″ to 4″ (2.5-10.2 cm).

When an additional string (not shown in the drawings) is to be attached to tampon string 16 without an accessory being used as well, the additional string is preferably 1″ to 12″ (2.5-30.5 cm), more preferably 2″ to 10″ (5.1-25.4 cm), and most preferably 3″ to 4″ (7.6-10.2 cm) long.

When an additional string 20 is added to string 16 (or when the tampon string is manufactured with a longer than customary string 116), the length of the longer than customary string 116 or the total length of the strings 16 and 20 together is preferably 3″ to 15″ (7.6-38.1 cm), more preferably 9″ to 13″ (22.9-33 cm), and most preferably 11″ to 12.5″ (27.9-31.8 cm).

Once the string 20 is added or the original string 116 lengthened it generally becomes long and may fall into the toilet if not properly handled. Therefore, the string is preferably shortened, temporarily, so that once the tampon 10 is inserted the accessory (tab 24 or 124, ring 25, tassel 26, or globe 28) can be pulled on and the string will lengthen. Any means can be used to temporarily shorten the string but allow it to become longer when pulled. Preferably, the string is tied into a bow so that when the accessory (tab 24, ring 25, tassel 26, or globe 28) is pulled the string lengthens. More preferably, the string is gathered together and tied or lightly glued so that once the accessory (tab 24, ring 25, tassel 26, or globe 28) is pulled the string lengthens.

Once the tampon 10 is inserted and the peel-off label is removed from one side of the tape making up tab 124, the woman may fold the tape back onto it self so that it forms a tab 124; this way the tampon string is easily found to make removal of the tampon easier. If the woman does not want to stick the tape to her skin or onto her underwear, she does not remove peel-off backing 30 or she can peel off the backing and fold the tape back onto itself to form a tab.

FIG. 14 shows the tampon with an enlarged knot 29.

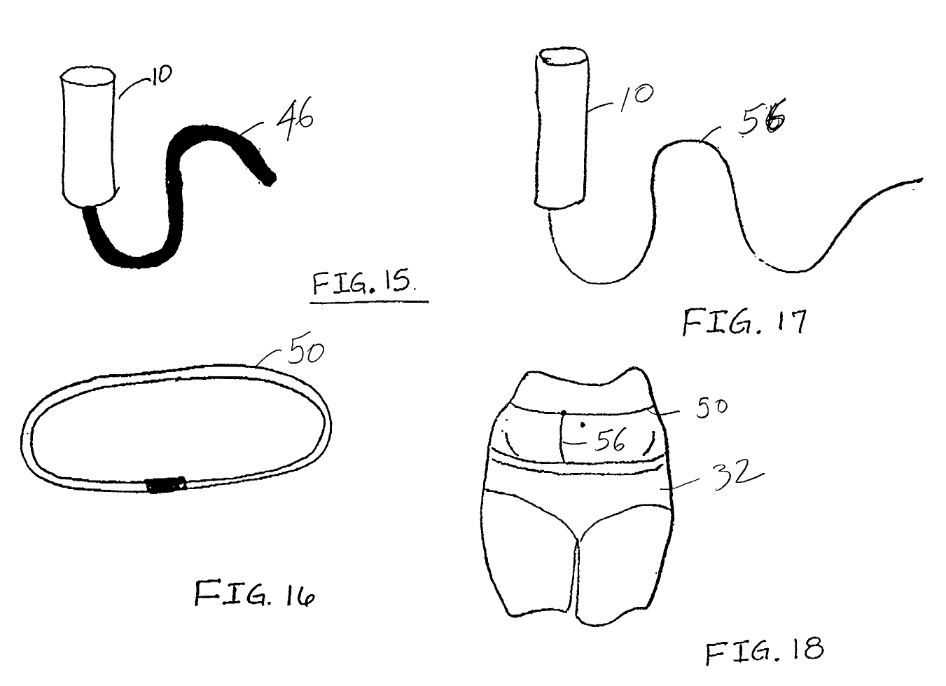

FIG. 15 shows the tampon 10 with a wide tampon string 46 (for example, it can be preferably ⅛″ to ½″ (0.32-1.3 cm) wide, more preferably ¼″ to ¾″ (0.64-1.9 cm) wide, and most preferably about 3/16″ (0.48 cm) wide).

FIG. 16 shows a waist band 50 to which the extra long tampon string 56 of FIG. 17 can attach (see FIG. 18).

Parts List

The following is a list of parts and materials suitable for use in the present invention:

tampon (such as a tampon currently commercially available in the United States)

insertion tube

plunger portion of insertion tube 12

tampon string attached to tampon 10

free, knotted end of string 16

extension string (preferably some type of nylon, cotton/nylon blend, or 100% cotton string,

more preferably some type of cotton/nylon blend or 100% cotton string, and most preferably

some type of 100% cotton string)

free end of extension string 20

capsule

tab

ring

tassel

knit globe or spherical body

enlarged knot

peel-off backing of a double sided, soft cloth tape

undergarment

wide tampon string

waist band

extra long tampon string

tampon apparatus of the preferred embodiment of the present invention

tampon string attached to tampon 10 of tampon apparatus 110

free, knotted end of string 116

tape tab

Additional and/or Alternative Specifications Lengths of Extension String

preferrably 1″ to 12″ (2.5-30.5 cm)

More preferably 3″ to 10″ (7.6-25.4 cm)

Most preferably 6″ (15.2 cm)

Types of Tape

Preferably a hypo allergenic tape

More preferably 3M's Microfoam tape

Most preferably Smith and Nephew's Hypafix Dressing Retention Sheet

Types of String

Preferably some type of nylon string

More preferably some type of cotton/nylon blend

Most preferably some type of 100% cotton string

Thickness of Tape

Preferably tape of any thickness

More preferably tape of 1/32″ thickness (0.8 mm)

Most preferably 1/128″ thickness (0.2 mm)

Length of Tape

Preferably ¼″ to 3″ (0.64-7.6 cm)

More preferably ½″ to 2″ (1.3-5.1 cm)

Most preferably ¾″ to 1½″ (1.9-3.8 cm)

Width of Tape

Preferably ¼″ to 3″ (0.64-7.6 cm)

More preferably ⅓″ to 2″ (0.85-5.1 cm)

Most preferably ½″ to 1″ (1.3-2.5 cm)

What to do with the String

Once the string is added or the original string lengthened it becomes too long and will fall into the toilet if not properly handled. Therefore, it needs to be shortened, temporarily, so that once the tampon is inserted the tab can be pulled on and the string will lengthen.

Preferably any way to temporarily shorten the string but when pulled on it becomes longer. More preferably to tie the string into a bow so that when the tab is pulled the string lengthens. Most preferably to have the string gathered together and tied or lightly glued so that once the tab is pulled the string lengthens.

Size of Pom-Pom

Preferably greater than 16 mm

More preferably 13 mm to 16 mm

Most preferably 10 mm to 13 mm

Type of Pom-Pom

Preferably made of nylon

More preferably made of acrylic

Most preferably made of cotton

Type of Tassel

Preferably made of nylon

More preferably made of acrylic

Most preferably made of cotton

Size of Ring

Preferably any size ring

More preferably ¾″ to 1½″ (1.9-3.8 cm) in diameter

Most preferably ½″ to 1″ (1.3-2.5 cm) in diameter

Type of Ring

The ring is preferably made out of any soft substance, more preferably made out of 3 M's Microfoam tape folded onto itself and cut into a ring, and most preferably made out of a satin cord.

The Tape Being made Into a Tab Only

Once the tampon is inserted and the peel-off label is removed from tape, if the woman does not want to stick the tape to her skin or onto her underwear, etc. she may fold the tape back onto itself so that it forms a “tab” this way the tampon string is easily found to make removal of the tampon easier.

To Enlarge the Knot on the Tampon String with no Tab, Pom-Pom, Tassel, or Ring

Preferably greater than 16 mm

More preferably 13 mm to 16 mm

Most preferably 10 mm to 13 mm

Instead of tape the manufacturer could use some type of putty or sticky substance which could be put onto the tampon string if the woman so desires. With this putty or other sticky substance the woman could stick this onto her skin or onto her underwear. The manufacturer may also choose to use wax or some other type of pliable substance which could be put onto the tampon string if the woman desires to have something on the end to better locate the tampon string.

Size of Putty or Wax

The manufacturer should include a 3″×3″×¼″ (7.6 cm×7.6 cm×0.64 cm) piece of the substance in the tampon box. This way the woman has the option to use it if would like. If she does choose to use it she can tear a small piece off and place it on the end of the tampon string.

The manufacturer may also opt to use a gel or saline filled capsule 23 on the end of the tampon string. The capsule 23 could be in any shape including round, like a pom-pom, or in the shape of a ring.

The capsule 23 is preferably made of rubber, more preferably made of plastic, and most preferably made of non-latex rubber with a fabric coating.

If the tampon string were to be made longer and wider the manufacturer could include a safety pin so that the woman could pin the tampon string to her underclothes.

If the tampon string were to be made longer and wider the manufacturer could put a sticky substance on the distal end of the tampon string, like what is used on a Band-Aid® brand bandage. The distal end of the tampon could then be rolled up so that once the tampon is inserted the end could be unrolled and the woman could stick the string where she wanted. The other option would be for the distal end, with the sticky substance, to be covered with paper, like what is on a Band-aid, so that once the tampon is inserted the paper can be removed and the woman could stick the string where she wanted.

It might also be possible to “coil” the tampon string, similar to those coiled shoe strings that kids wear.

If the tampon string were made longer it could be connected to a belt-type apparatus which would go around the woman's stomach, hips or leg.

All measurements disclosed herein are at standard temperature and pressure, at sea level on Earth, unless indicated otherwise. All materials used or intended to be used in a human being are biocompatible, unless indicated otherwise.

The foregoing embodiments are presented by way of example only; the scope of the present invention is to be limited only by the following claims.

1. Apparatus including:
a tampon having a tampon string, the tampon string having a first end attached to a tampon and a second, free end distal from the tampon; and an attachment for the tampon string, the attachment comprising an accessory secured to the free end of the tampon string, the accessory providing a means for locating the free end of the tampon string and/or for preventing the free end of the tampon string from being retracted into the vagina of a user of the tampon, wherein the accessory is a length of tape forming a tab secured to the free end of the tampon string, the tape having a sticky surface.

2. The apparatus according to claim 1 wherein the tab comprises a peel-off backing which, when removed, exposes the sticky surface.

3. Apparatus including a tampon having a tampon string having an end and a tab comprising a piece of tape secured to or adjacent the end of the tampon string, the tape having a sticky surface.

4. The apparatus according to claim 3 wherein said tab further comprises a peel-off backing which, when removed, exposes the sticky surface.

5. The apparatus according to claim 3 wherein said tab is a piece of tape ½ to 2 inches long and ½ to 2 inches wide secured to the string.

6. The apparatus according claim 3 wherein said tab is apiece of hypo-allergenic tape ½ to 2 inches wide and ½ to 2 inches long secured to the string.

7. The apparatus according to claim 3 wherein said tab is a piece of tape which can be rolled into a cylindrical shape and unrolled to allow the use of portions of tape.

8. Apparatus including:
a tampon having a tampon string, the tampon string having a first end attached to a tampon and a second, free end distal from the tampon; and
an attachment for the tampon string, the tampon string having a first end attached to a tampon and a second, free end distal from the tampon, the attachment comprising a tampon extension string having an accessory secured thereto, the attachment being securable to the free end of the tampon string, the accessory providing a means for locating the free end of the tampon string and/or for preventing the free end of the tampon string from being retracted into the vagina of a user of the tampon.

9. The apparatus according to claim 8 wherein the accessory is a tab comprising a length of tape secured around an end of the tampon extension string.

10. The apparatus according to claim 8 wherein the accessory is a tassel secured to the extension string.

11. The apparatus according to claim 8 wherein the accessory is a soft, hollow, spherical body having a polar axis and which is collapsible upon axial compression along its polar axis.

12. The apparatus according to claim 8 wherein the accessory is a tab comprising a length of tape secured around an end of the tampon extension string, and the tab comprises a peel-off backing which, when removed, exposes a sticky surface.

13. A method for altering a tampon to enhance a tampon user's ability to locate and manipulate a tampon string during urination and aid in the prevention of tampon string retraction into the user's vagina, the tampon having a tampon string with a free end distal from the tampon, comprising the steps of attaching an accessory to the free end of the tampon string, the accessory providing a means for locating the free end of the tampon string and/or for preventing the free end of the tampon string from being retracted into the vagina of a user of the tampon.

14. The method according to claim 13 further comprising the steps of providing an extension string attached to the accessory and securing the extension string to the tampon string.

15. The method according to claim 13 further comprising the step of adhering the accessory to a portion of the user's body or clothing.

16. The method according to claim 13 further comprising the step of extending the length of a tampon string to between 3 and 11 inches (7.6 and 27.9 cm) long.

17. The method according to claim 14 further comprising the step of adhering the accessory to a portion of the user's body or clothing.

18. The method according to claim 11 wherein the accessory is a tab comprising a length of tape secured around an end of the tampon extension string.

19. The apparatus according to claim 11 wherein the accessory is a tab comprising a length of tape secured around an end of the tampon extension string, and the tab comprises a peel-off backing which, when removed, exposes a sticky surface.

20. Apparatus comprising:
a tampon;
a tampon string attached to the tampon, the tampon string having a first end attached to a tampon and a second, free end distal from the tampon, the tampon string having a length of 3″ to 15″ (7.6-38.1 cm).

21. The apparatus of claim 20, wherein the tampon string has a length of 9″ to 13″ (22.9-33 cm).

22. The apparatus of claim 20, wherein the tampon string has a length of 11″ to 12.5″ (27.9-31.8 cm).

23. Apparatus comprising:
a tampon;
a tampon string attached to the tampon, the tampon string having a first end attached to a tampon and a second, free end distal from the tampon, the tampon string having a width of ⅛″ to ½″ (0.32-1.3 cm).

24. The apparatus of claim 23, wherein the tampon string has a width of ¼″ to ¾″ (0.64-1.9 cm).

25. The apparatus of claim 23, wherein the tampon string has a width of about 3/16″ (0.48 cm).

26. The apparatus of one of claims 23-25, further comprising an accessory attached adjacent the free end of the tampon string.

27. The invention according to one of claims 1-19 and 26, wherein the accessory includes a piece of tape forming a tab secured to the free end of the tampon string, and the tab comprises a peel-off backing which, when removed, exposes a sticky surface.

28. The invention of one of claims 1-19, 26, and 27, wherein the tampon string has a length sufficient to allow the accessory to protrude from the underwear of a user.

29. The invention substantially as shown and described herein.
